(19) United States
(12) Patent Application Publication
(10) Pub. No.: US 2013/0230460 A1
(43) Pub. Date: Sep. 5, 2013

(54) USE OF CYANINE DYES FOR THE DETECTION OF TAU FOR DIAGNOSIS OF EARLY-STAGE TAUOPATHIES

(71) Applicants: Clare Jones, Amersham (GB); James Nairne, Amersham (GB)

(72) Inventors: Clare Jones, Amersham (GB); James Nairne, Amersham (GB)

(73) Assignee: GE HEALTHCARE LIMITED, BUCKINGHAMSHIRE (GB)

(21) Appl. No.: 13/885,825

(22) PCT Filed: Nov. 15, 2011
(86) PCT No.: PCT/US11/60737

§ 371 Date: May 16, 2013

Related U.S. Application Data
(60) Provisional application No. 61/415,366, filed on Nov. 19, 2010.

(51) Int. Cl. C07D 417/06

(52) U.S. Cl. CPC C07D 417/06
USPC 424/1.89; 548/156; 435/7.1

(57) ABSTRACT

Radiolabeled compounds useful as diagnostic imaging agents of Tau pathology in Alzheimer's disease are described. Compositions and methods of making such compounds are also described.

### TECHNICAL FIELD OF THE INVENTION

The present invention relates to radiolabeled compounds, compositions thereof, methods of making such compounds and their use as imaging probes of Tau pathology especially as it relates to Alzheimer's Disease. Compounds of the present invention may be used for Positron Emission Tomography (PET) or Single Photon Emission Computed Tomography (SPECT) imaging.

### DESCRIPTION OF RELATED ART

Alzheimer's disease (AD) is the most common cause of dementia in the elderly. It is definitively diagnosed and staged on the basis of post-mortem neuropathology. The pathological hallmark of AD is a substantial neuronal loss accompanied by deposition of amyloid plaques and neurofibrillary tangles (NFTs).

NFTs consist of filamentous aggregates composed of microtubule-associated protein tau. Much of the literature suggests that tau aggregates (NFTs) or NFT formation correlate more closely with AD progression than amyloid plaques (Braak, H. et al., Neuropathological Staging of Alzheimer-related Changes. Acta Neuropathologica, 82, 239-259, 1991). The tau aggregates or neurofibrillary lesions reportedly appear in areas (deep temporal lobe) decades before neocortical amyloid deposition and signs of dementia can be detected. The tau lesions occur before the presentation of clinical symptoms or signs of dementia and correlate with the severity of dementia. These attributes make tau aggregates a potentially superior approach for the early diagnosis of AD. Hence in vivo detection of these lesions or NFTs would prove useful for diagnosis of AD and for tracking disease progression.

Thiacarbocyanines possess tau antagonist activity. Symmetrical thiacarbocyanines, such as N744, are potent tau aggregation inhibitors:

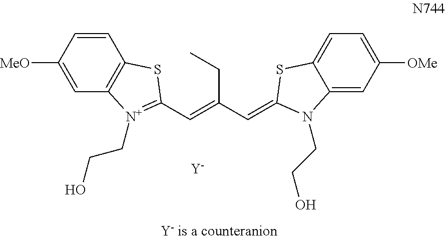

(E Chang, E E Congdon, N S Honson, K E Duff, and J Kuret, J Med Chem, 2009, 52, 3539). Cyanine dyes:

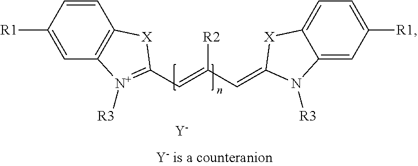

in particular benzothiazolyl trimethine cyanines, have been reported to inhibit the formation of insoluble tau aggregates at low concentrations (<300 nM)(E Chang, E E Congdon, N S Honson, K E Duff, and J Kuret, J Med Chem, 2009, 52, 3539).

However there still exists a need in the art for a method of preparing radiolabeled compounds that can be used as imaging agents for NFTs. The present invention described below answers such a need.

### BRIEF DESCRIPTION OF THE DRAWINGS

FIG. 1 depicts HPLC analysis of a reaction mixture containing [¹⁸F]9 (8.0 min), top: radioactivity channel, bottom: UV channel at 254 nm.

FIG. 2 depicts HPLC analysis of a reaction mixture containing [¹⁸F]9 (7 8 min) with added ¹⁹F-20 reference compound (7.8 min), top: radioactivity channel, bottom: UV channel at 254 nm

FIG. 3 depicts HPLC analysis of a reaction mixture containing [¹⁸F]9 (7.9 min), top: radioactivity channel, bottom: UV channel at 254 nm.

### SUMMARY OF THE INVENTION

The present invention provides a radiolabeled compound of Formula (I):

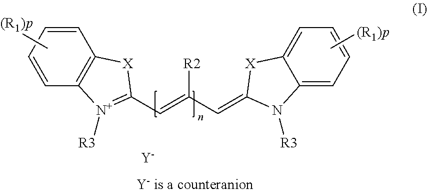

wherein:
R₁ are each independently H, alkyl, alkoxy, hydroxyalkyloxy, or hydroxy poly(oxoethylene)oxy;
p are each independently an integer from 1-4;
X are each independently N, O or S; preferably S;
R2 is H or alkyl;
R3 are each independently H, alkyl, hydroxyalkyl or hydroxy poly(oxoethylene);
n is an integer from 1-5; preferably, 1-4; more preferably, 1-3; and
Y⁻ is any counteranion known in the art; preferably I⁻, Br⁻, Cl⁻, triflate, trifluoroacetate, tosylate, or mesylate; more preferably, I⁻;
and wherein at least one of R1, R2, and R3 contains a radionuclide.

The present invention provides a radiolabeled compound of Formula (Ia):

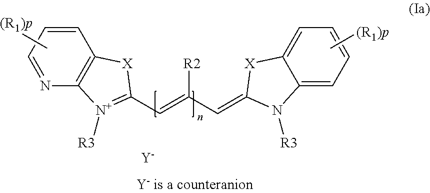

wherein:
R₁ are each independently H, alkyl, alkoxy, hydroxyalkyloxy, or hydroxy poly(oxoethylene)oxy;
p are each independently an integer from 1-4;
X are each independently N, O or S; preferably S;
R2 is H or alkyl;
R3 are each independently H, alkyl, hydroxyalkyl or hydroxy poly(oxoethylene);
n is an integer from 1-5; preferably, 1-4; more preferably, 1-3; and
Y⁻ is any counteranion known in the art; preferably, I⁻, Br⁻, Cl⁻, triflate, trifluoroacetate, tosylate, or mesylate; more preferably, I⁻;
and wherein at least one of R1, R2, and R3 contains a radionuclide.

The present invention provides a radiolabeled compound of Formula (II):

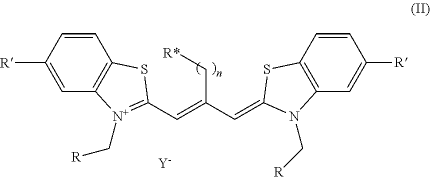

wherein:
R are each independently H, alkyl, hydroxyalkyl or hydroxy poly(oxoethylene);
R′ are each independently H, alkyl, alkoxy, hydroxyalkyloxy, or hydroxy poly(oxoethylene)oxy;
n is an integer from 1-6;
R* is a radionuclide; preferably ¹⁸F; and
Y⁻ is any counteranion known in the art; preferably, I⁻, Br⁻, Cl⁻, triflate, trifluoroacetate, tosylate, or mesylate; more preferably, I⁻.

The present invention provides a radiolabeled compound of Formula (IIa):

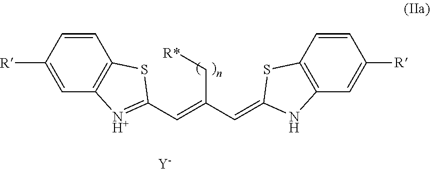

wherein R′, n, R*, and Y⁻ are each as defined for a compound of Formula (II).

The present invention provides a radiolabeled compound of Formula (III):

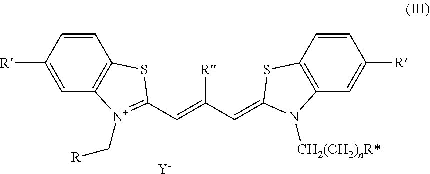

wherein:

R is H, alkyl, hydroxyalkyl or hydroxy poly(oxoethylene);

R′ are each independently H, alkyl, alkoxy, hydroxyalkyloxy, or hydroxy poly(oxoethylene)oxy;

R″ is H or alkyl;

R* is a radionuclide; preferably ¹⁸F;

n is an integer from 1-10; preferably, 1-7; more preferably, 1-5; and

Y⁻ is any counteranion known in the art; preferably I⁻, Br⁻, Cl⁻, triflate, trifluoroacetate, tosylate, or mesylate; more preferably, I⁻.

The present invention provides a radiolabeled compound of Formula (IV):

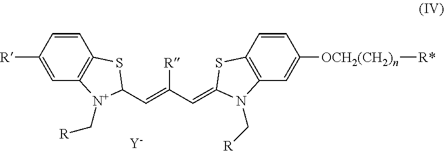

wherein:

R are each independently H, alkyl, hydroxyalkyl or hydroxy poly(oxoethylene);

R′ is H, alkyl, alkoxy, hydroxyalkyloxy, or hydroxy poly(oxoethylene)oxy;

R″ is H or alkyl;

R* is a radionuclide; preferably ¹⁸F;

n is an integer from 1-10; preferably, 1-7; more preferably, 1-5; and

Y⁻ is any counteranion known in the art; preferably I⁻, Br⁻, Cl⁻, triflate, trifluoroacetate, tosylate, or mesylate; more preferably, I⁻.

The present invention provides a radiolabeled compound of Formula (IVa):

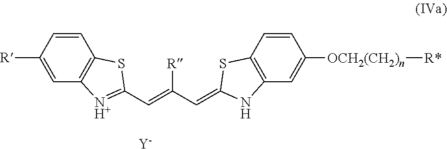

wherein R′, R″, R*, n and Y⁻ are each as defined for a compound of Formula (IV).

The present invention provides a method of preparing a radiolabeled compound of Formulae (I), (Ia), (II), (IIa), (III), (IV) and/or (IVa).

The present invention further provides a pharmaceutical composition comprising a compound of Formulae (I), (Ia), (II), (IIa), (III), (IV) or (IVa) and a pharmaceutically acceptable carrier or excipient.

The present invention provides a method of detecting tau aggregates or NFTs in vitro or in vivo by administering a radiolabeled compound of the invention each as described herein or a pharmaceutical composition thereof.

The present invention provides a method of imaging comprising the step of administering a radiolabeled compound of the invention each as described herein or a pharmaceutical composition thereof.

The present invention provides radiolabeled compounds for use as diagnostic imaging agents for the presymptomatic detection of Alzheimer's disease and other tauopathies. The compounds of the inventions may be radiolabeled such that they may be used for in vitro and in vivo imaging purposes.

### DETAILED DESCRIPTION OF THE INVENTION

Examples of a compound of the present invention include:

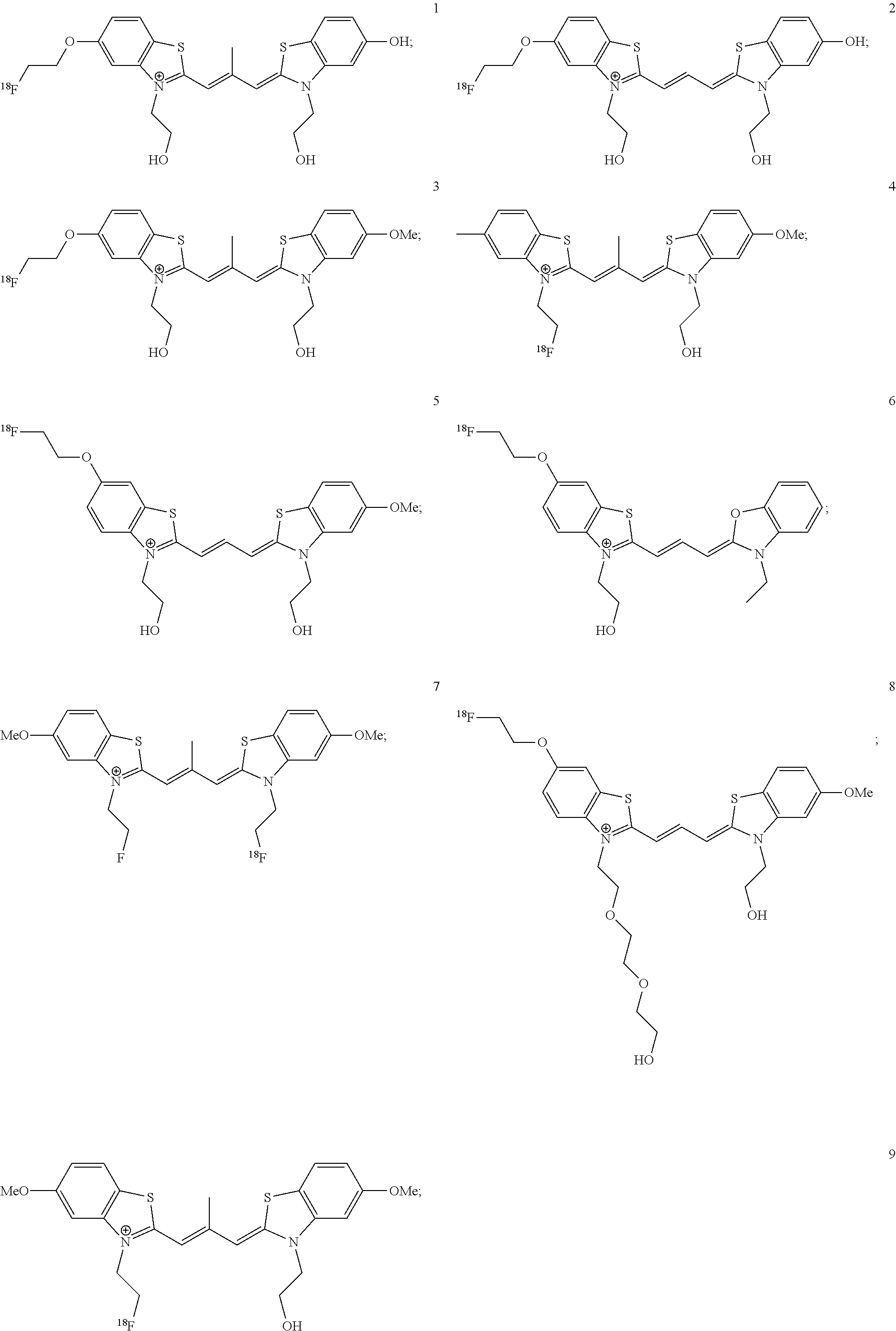

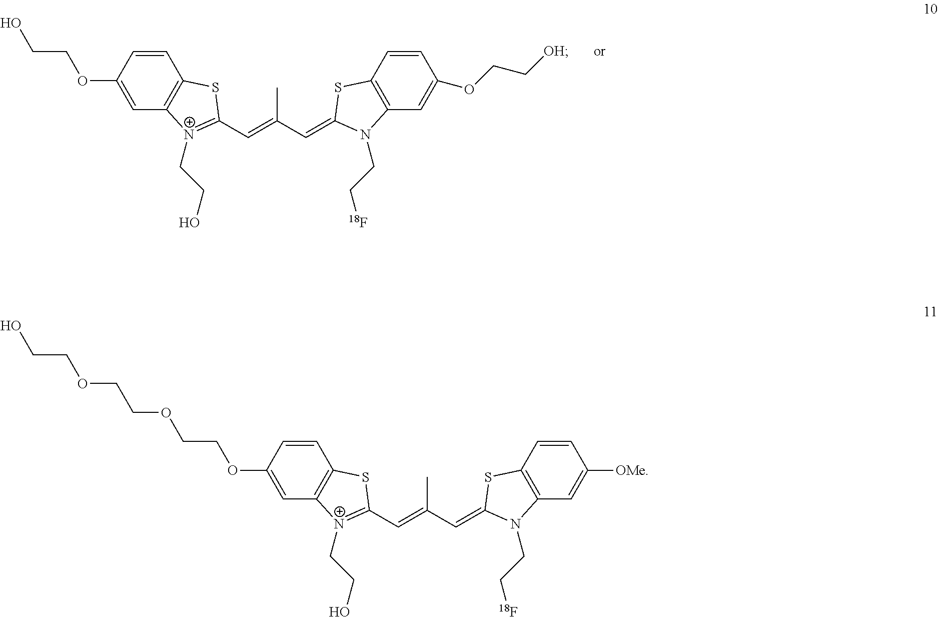 [SMILES: CC(/C=C1\Sc2ccc(OCCO)cc2N1CC[18F])=C\c1sc2ccc(OCCO)cc2[n+]1CCO ; COc1ccc2c(c1)N(CC[18F])/C(=C/C(C)=C/c1sc3ccc(OCCOCCOCCO)cc3[n+]1CCO)S2]

According to the present invention, for a compound of the invention described herein, a radionuclide shall mean any radioisotope known in the art (hereinafter referred to as a “radiolabeled compound”). Preferably the radionuclide is a radioisotope suitable for imaging (e.g., PET, SPECT). In one embodiment, the radionuclide is a radioisotope suitable for PET imaging. Even more preferably, the radionuclide is ¹¹C, ¹³N, ¹⁵O, ⁶⁸Ga, ⁶²Cu, ¹⁸F, ⁷⁶Br, ¹²⁴I, or ¹²⁵I; even more preferably, the radionuclide is ¹⁸F.

In one embodiment, the radionuclide is a radioisotope suitable for SPECT imaging. Even more preferably, the radionuclide is ^{99m} Tc, ₁₁₁In, ⁶⁷Ga, ²⁰¹T1, ¹²³I, or ¹³³Xe; even more preferably, the radionuclide is ^{99m}Tc or ¹²³I.

The present invention provides a compound of Formulae (I), (Ia), (II), (IIa), (III), (IV) and/or (IVa) wherein the radionuclide is replaced with its non-radioisotopic equivalent (e.g., ¹⁸F is replaced with ¹⁹F or F)(”non-radiolabeled compounds“).

The present invention provides a method of making non-radiolabeled compounds of Formulae (I), (Ia), (IIa), (III), (IV) and/or (IVa).

The present invention provides a pharmaceutical composition comprising a non-radiolabeled compound of Formulae (I), (Ia), (IIa), (III), (IV) and/or (IVa) and a pharmaceutically acceptable carrier or excipient.

Examples of non-radiolabeled compounds of Formulae (I), (Ia), (II), (IIa), (III), (IV) and/or (IVa) include:

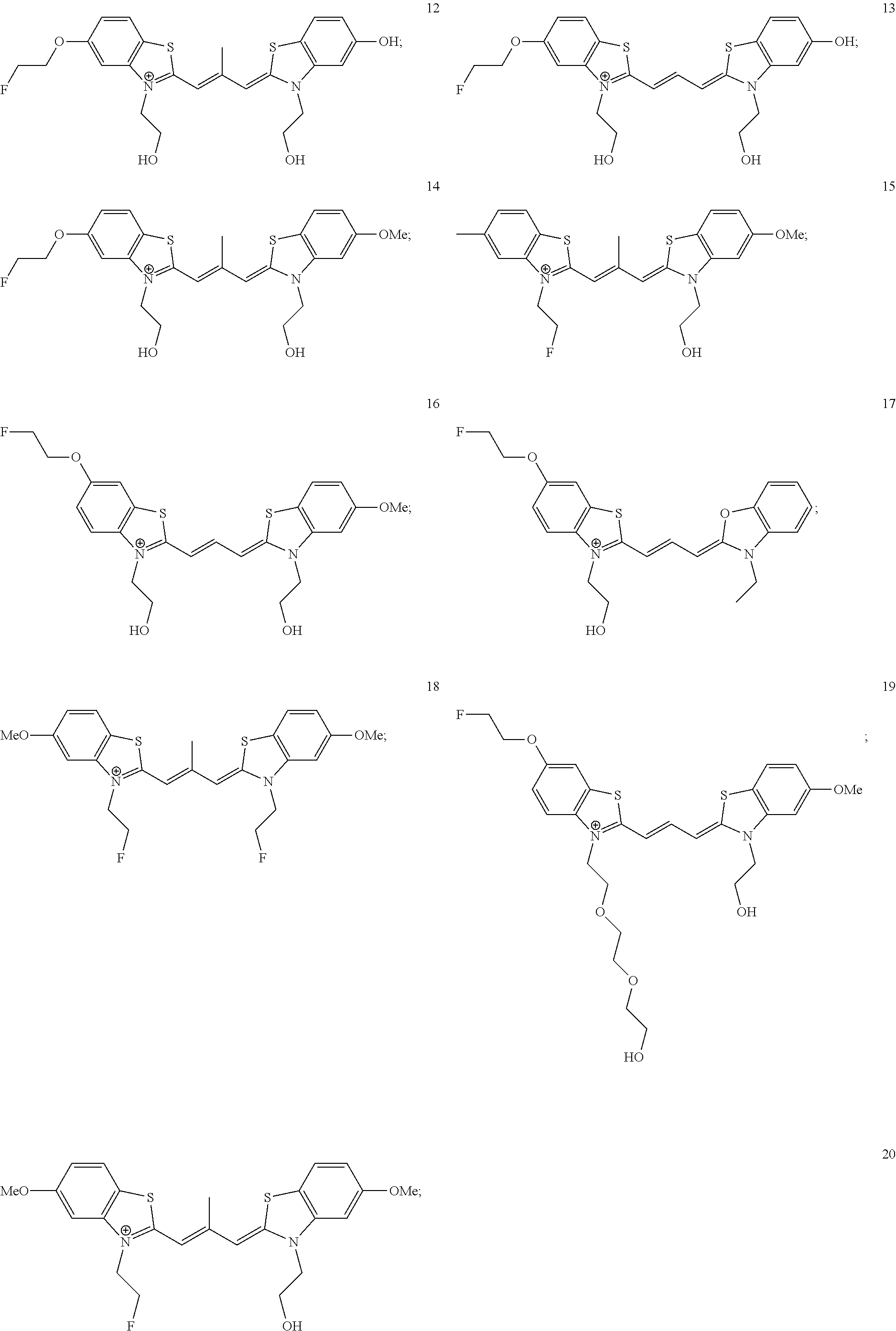

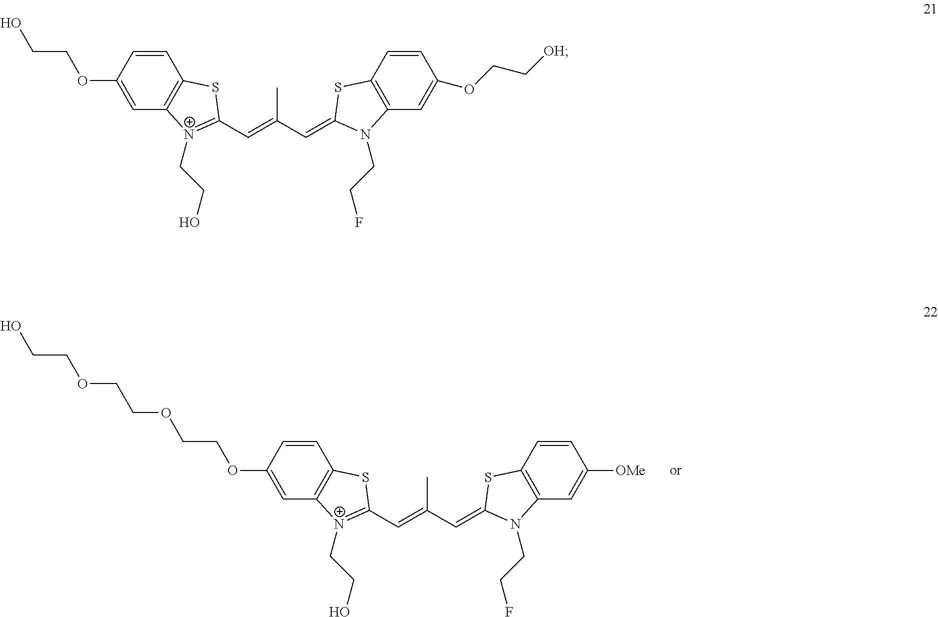 [SMILES: CC(/C=C1\Sc2ccc(OCCO)cc2N1CCF)=C\c1sc2ccc(OCCO)cc2[n+]1CCO ; COc1ccc2c(c1)N(CCF)/C(=C/C(C)=C/c1sc3ccc(OCCOCCOCCO)cc3[n+]1CCO)S2]

### Pharmaceutical or Radiopharmaceutical Composition

The present invention provides a pharmaceutical or radiopharmaceutical composition comprising a compound of the invention as described herein together with a pharmaceutically acceptable carrier, excipient, or biocompatible carrier. According to the invention when a compound of the invention is radiolabeled with a radionuclide, the pharmaceutical composition is a radiopharmaceutical composition.

The present invention further provides a pharmaceutical or radiopharmaceutical composition comprising a compound of the invention as described herein together with a pharmaceutically acceptable carrier, excipient, or biocompatible carrier suitable for mammalian administration.

As would be understood by one of skill in the art, the pharmaceutically acceptable carrier or excipient can be any pharmaceutically acceptable carrier or excipient known in the art.

The “biocompatible carrier” can be any fluid, especially a liquid, in which a compound of the invention can be suspended or dissolved, such that the pharmaceutical composition is physiologically tolerable, e.g., can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g., salts of plasma cations with biocompatible counterions), sugars (e.g., glucose or sucrose), sugar alcohols (e.g., sorbitol or mannitol), glycols (e.g., glycerol), or other non-ionic polyol materials (e.g., polyethyleneglycols, propylene glycols and the like). The biocompatible carrier may also comprise biocompatible organic solvents such as ethanol. Such organic solvents are useful to solubilise more lipophilic compounds or formulations. Preferably the biocompatible carrier is pyrogen-free water for injection, isotonic saline or an aqueous ethanol solution. The pH of the biocompatible carrier for intravenous injection is suitably in the range 4.0 to 10.5.

The pharmaceutical or radiopharmaceutical composition may be administered parenterally, i.e., by injection, and is most preferably an aqueous solution. Such a composition may optionally contain further ingredients such as buffers; pharmaceutically acceptable solubilisers (e.g., cyclodextrins or surfactants such as Pluronic, Tween or phospholipids); pharmaceutically acceptable stabilisers or antioxidants (such as ascorbic acid, gentisic acid or para-aminobenzoic acid). Where a compound of the invention is provided as a radiopharmaceutical composition, the method for preparation of said compound may further comprise the steps required to obtain a radiopharmaceutical composition, e.g., removal of organic solvent, addition of a biocompatible buffer and any optional further ingredients. For parenteral administration, steps to ensure that the radiopharmaceutical composition is sterile and apyrogenic also need to be taken. Such steps are well-known to those of skill in the art.

### Preparation of a Compound of the Invention

A compound of the invention may be prepared by any means known in the art including, but not limited to, nucleophilic aromatic substitution, nucleophilic aliphatic substitution, and click chemistry.

Examples by which a compound of the invention may be prepared is the reaction of two N-alkylated 2-methylheterocycles (e.g., 23a and 23b) with either an amidine (24)(Scheme A) or an orthoester (25) (Scheme B).

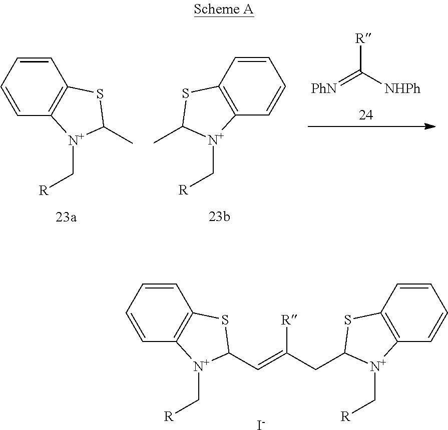

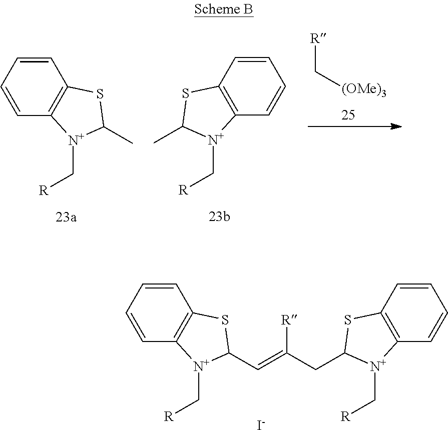

In Schemes A and B, R and R″ are each as defined herein.

In one embodiment of the invention, a compound of the invention may be radiolabeled with a radionuclide by nucleophilic aromatic substitution or nucleophilic aliphatic substitution of an appropriate leaving group with the desired halogen or radionuclide. Examples of suitable leaving groups for nucleophilic aromatic substitution include, but are not limited to, Cl, Br, F, NO₂ and ⁺N(R)₄. Examples of suitable leaving groups for nucleophilic aliphatic substitution include, but are not limited to, I, Br, Cl, OMs (mesylate) and OTs (tosylate).

In one embodiment of the invention, the radionuclide will be introduced in a one-step radiosynthesis. By way of example, a compound of Formula (II) can be radiolabeled with ¹⁸F according to Scheme C (where R is as described herein) below:

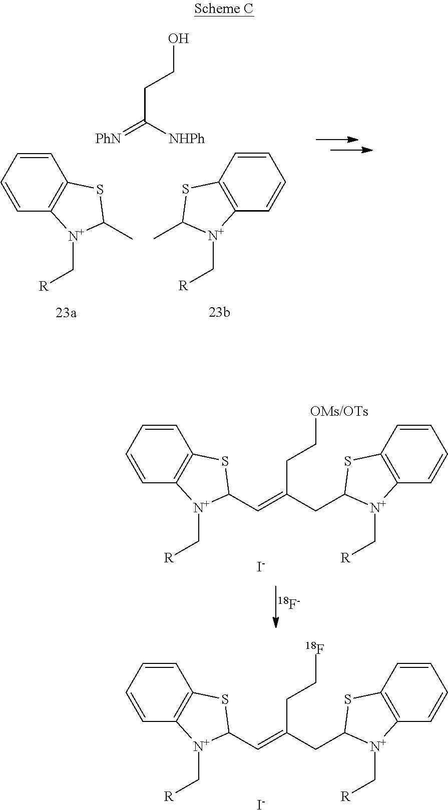

Compounds of Formulae (III) and (IV) may also be prepared according to Scheme C except that 23a and 23b would not be symmetrical.

Schemes D, E, F and G illustrates various routes for radiolabeling the aromatic ring of a compound of the invention (R is as described herein):

Scheme D. Use of pyridine rings for nucleophilic fluoridation may be accomplished by means known in the art. The nitrogen of the pyridyl moiety makes the ring more electron-withdrawing and hence promotes fluorination. The leaving group (LG) will be ortho or para with respect to the nitrogen of the pyridyl moiety.

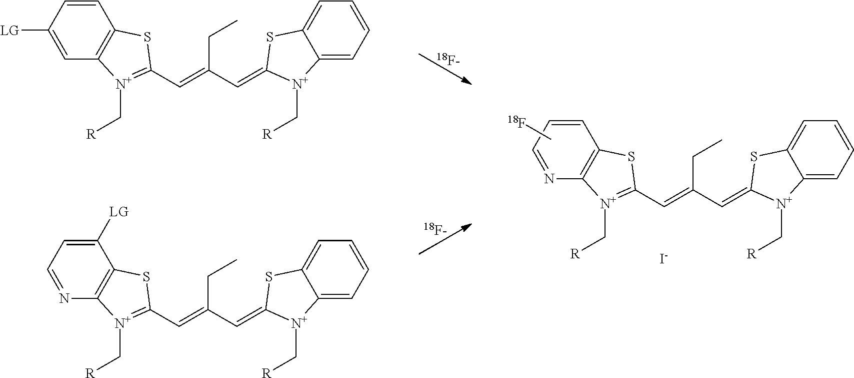

Scheme E. Tobias Ritter (Takeru Furuya, Hanns Martin Kaiser, Angew. Chem. Int. Ed., 2008, 47, 5993-5996):

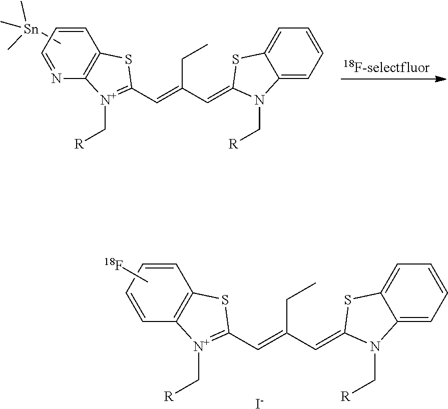

Scheme F. Buchwald method (Donald A. Watson, Mingjuan Su, Georgiy Teverovsky, Yong Zhang, Jorge Garcia-Fontanet, Tom Kinzel, Stephen L. Buchwald, Science, V325, 2009, 1661).

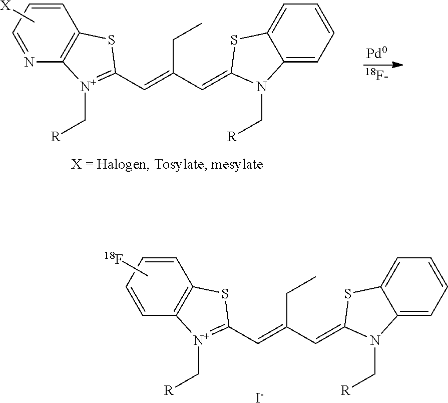

Scheme G. Use of iodinium salt as leaving group (WO2005097713 A1; WO2005061415 A1)

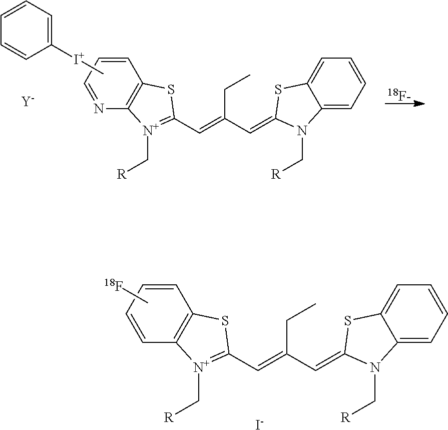

By way of example, the radioisotope [¹⁸M-fluoride ion (¹⁸F⁻) is normally obtained as an aqueous solution from the nuclear reaction ¹⁸O(p,n)¹⁸F and is made reactive by the addition of a cationic counterion and the subsequent removal of water. Suitable cationic counterions should possess sufficient solubility within the anhydrous reaction solvent to maintain the solubility of 18F⁻. Therefore, counterions that have been used include large but soft metal ions such as rubidium or caesium, potassium complexed with a cryptand such as Kryptofix™, or tetraalkylammonium salts. A preferred counterion is potassium complexed with a cryptand such as Kryptofix™ because of its good solubility in anhydrous solvents and enhanced ¹⁸F⁻ reactivity. ¹⁸F can also be introduced by nucleophilic displacement of a suitable leaving group such as a halogen or tosylate group. A more detailed discussion of well-known ¹⁸F labelling techniques can be found in Chapter 6 of the “Handbook of Radiopharmaceuticals” (2003; John Wiley and Sons: M. J. Welch and C. S. Redvanly, Eds.). Similar methods may be used to radiolabel a compound of the invention with other radioisotopes including the PET and SPECT radioisotopes described herein.

### Automated Synthesis

In one embodiment, the method to prepare a radiolabeled compound of the invention, each as described herein, is automated. For example, [¹⁸F]-labeled compounds of the invention may be conveniently prepared in an automated fashion by means of an automated radiosynthesis apparatus. There are several commercially-available examples of such platform apparatus, including TRACERlab™ (e.g., TRACERlab™ MX) and FASTlab™ (both from GE Healthcare Ltd.). Such apparatus commonly comprises a “cassette”, often disposable, in which the radiochemistry is performed, which is fitted to the apparatus in order to perform a radiosynthesis. The cassette normally includes fluid pathways, a reaction vessel, and ports for receiving reagent vials as well as any solid-phase extraction cartridges used in post-radiosynthetic clean up steps. Optionally, in a further embodiment of the invention, the automated radiosynthesis apparatus can be linked to a high performance liquid chromatograph (HPLC). The present invention therefore provides a cassette for the automated synthesis of a compound of the invention.

### Imaging Method

The radiolabeled compound of the invention, as described herein, may bind to NFTs or tau aggregates and aid in identifying the amount of NFTs/tau aggregates present which in turn may correlate with the stage of AD (see e.g., Modulation and detection of tau aggregation with small-molecule ligands, Current Alzheimer Research, 2009, 6, 409-414, Edward Chang, Nicolette S. Honson, Kristen E. Funk, Jordan R. jensen, Bhaswati Bandyopadhyay, Sohee Kim, Swati Naphade and Jeff Kuret).

The present invention further provides a method of imaging comprising the step of administering a radiolabeled compound of the invention, as described herein, to a subject and detecting said radiolabeled compound of the invention in said subject. The present invention further provides a method of detecting tau aggregates in vitro or in vivo using a radiolabeled compound of the invention, as described herein. The radiolabeled compound of the invention is preferably administered as a radiopharmaceutical composition of the invention, as defined herein. Hence the present invention provides better tools for early detection and diagnosis of Alzheimers disease.

As would be understood by one of skill in the art the type of imaging (e.g., PET, SPECT) will be determined by the nature of the radioisotope. For example, if the radiolabeled compound of the invention contains ¹⁸F it will be suitable for PET imaging.

Thus the invention provides a method of detecting tau aggregates in vitro or in vivo comprising the steps of:
i) administering to a subject a radiolabeled compound of the invention as defined herein;
ii) allowing said a radiolabeled compound of the invention to bind to NFTs in said subject;
iii) detecting signals emitted by said radioisotope in said bound radiolabeled compound of the invention;
iv) generating an image representative of the location and/or amount of said signals; and,
v) determining the distribution and extent of said tau aggregates in said subject.

The step of “administering” a radiolabeled compound of the invention is preferably carried out parenterally, and most preferably intravenously. The intravenous route represents the most efficient way to deliver the compound throughout the body of the subject. Intravenous administration neither represents a substantial physical intervention nor a substantial health risk to the subject. The administration step is not required for a complete definition of the imaging method of the invention. As such, the imaging method of the invention can also be understood as comprising the above-defined steps (ii)-(v) carried out on a subject to whom a radiolabeled compound of the invention has been pre-administered.

Following the administering step and preceding the detecting step, the radiolabeled compound of the invention is allowed to bind to the tau aggregates. For example, when the subject is an intact mammal, the radiolabeled compound of the invention will dynamically move through the mammal's body, coming into contact with various tissues therein. Once the radiolabeled compound of the invention comes into contact with the tau aggregates it will bind to the tau aggregates.

The “detecting” step of the method of the invention involves detection of signals emitted by the radioisotope comprised in the radiolabeled compound of the invention by means of a detector sensitive to said signals, e.g., a PET camera. This detection step can also be understood as the acquisition of signal data.

The “generating” step of the method of the invention is carried out by a computer which applies a reconstruction algorithm to the acquired signal data to yield a dataset. This dataset is then manipulated to generate images showing the location and/or amount of signals emitted by the radioisotope. The signals emitted directly correlate with the amount of enzyme or neoplastic tissue such that the “determining” step can be made by evaluating the generated image.

The “subject” of the invention can be any human or animal subject. Preferably the subject of the invention is a mammal. Most preferably, said subject is an intact mammalian body in vivo. In an especially preferred embodiment, the subject of the invention is a human.

The “disease state associated with the tau aggregates” can be MCI (mild cognitive impairment), dementia or Alzheimers disease.

### EXAMPLES

Unless stated otherwise, all reagents and equipment are commercially available.

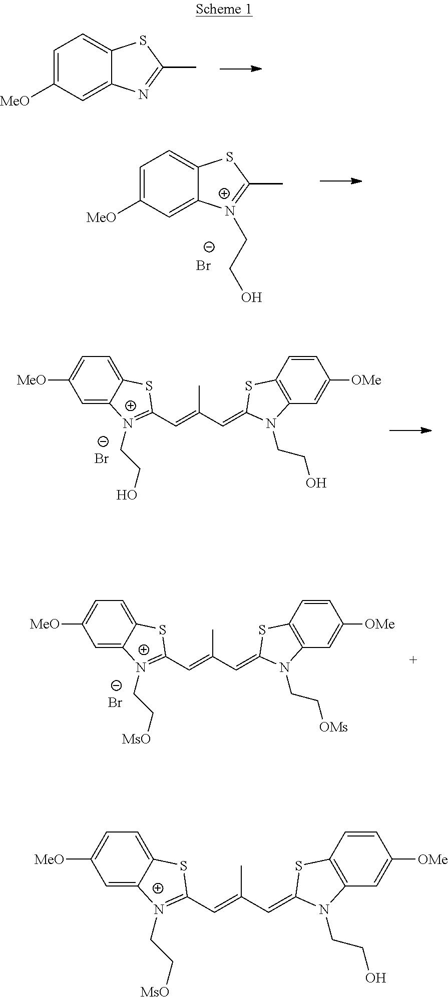

### Example 1

### Preparation of N-Hydroxyethyl-5-methoxy-2-methylbenzothiazolium bromide

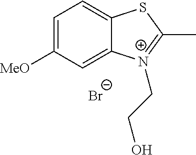

A mixture of 5-methoxy-2-methylbenzothiazole (5 g, 27 9 mmol) and 2-bromoethanol (5.22 g, 41.9 mmol, 2.96 mL) was heated at 120 C for 12 h. The resulting mixture was washed with CHCl₃ to remove the excess of bromoethanol and filtered to get the pure product (7 g)(83%) product as an off-white solid.

LC-MS: m/z calcd for C₁₁H₁₄Br⁻N⁺O₂S 304.2, found 223.8.

¹H NMR (500 MHz, DMSO-d₆): δ3.20 (3H, s, CH₃C), 3.90 (2H, t, NCH₂CH₂OH, J=5 Hz), 3.96 (3H,s, OCH₃), 4.86 (2H, t, NCH₂CH₂OH, J=5 Hz), 7.43 (1H, dd, Ar-4-CH, J=5,10 Hz), 7.82 (1H, d,Ar-6-CH, J=5 Hz), 8.32 (1H, d, Ar-7-CH, J=10 Hz).

### Example 2

### Preparation of 3-(2-Hydroxyethyl)-2-((1E,3Z)-3-(3-(2-hydroxyethyl)-5-methoxybenzo[d]thiazol-2(3H)-ylidene)-2-methylprop-1-en-1-yl)-5-methoxybenzo[d]thiazol-3-ium bromide

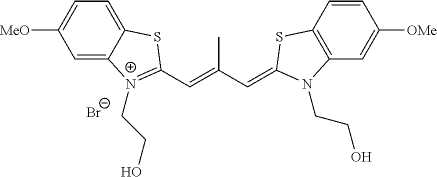 [SMILES: COc1ccc2c(c1)N(CCO)/C(=C/C(C)=C/c1sc3ccc(OC)cc3[n+]1CCO)S2 ; [Br-]]

A mixture of N-Hydroxyethyl-5-methoxy-2-methylbenzothiazolium bromide (see Example 1; 6.5 g, 21.36 mmol) and triethyl orthoacetate (1.73 g, 10.68 mmol) in pyridine (65 mL) was heated at 120° C. for 12 h. The completion of the reaction was checked by LCMS. More triethyl orthoacetate (1.73 g, 10.68 mmol) was added and the reaction was heated for another 12 h. The reaction mass was quenched by the addition of the diethyl ether(30 mL).The supernatant solution was separated and the solid formed were recrystallized from methanol and acetone to get 3.6 g (32%) of the dark pink colour solid.

LC-MS: m/z calcd for C₂₄H₂₇Br⁻N⁺₂O4S₂ 551.52, found 470.4

¹H NMR (500 MHz, DMSO-d₆): δ2.57 (3H, s,CHC(CH₃)CH), 3.90 (8H, s, 2-OCH₃, NCH₂CH₂OH), 4.55(4H, t, NCH₂CH₂OH, J=5 Hz), 5.13 (2H, t, NCH₂CH₂0H, J=5 Hz), 6.60 (2H, s, CHC(CH₃)CH), 7.07(2H, m, 2-Ar-4-CH), 7.37 (2H, s, 2-Ar-6-CH), 7.93 (2H, d, 2-Ar-7-CH, J=10 Hz)

### Example 3

### Preparation of Methoxy-2-((1E,3Z)-3-(5-methoxy-3-(2-((methylsulfonyl)oxy)ethyl)benzo[d]thiazol-2(3H)-ylidene)-2-methylprop-1-en-1-yl)-3-(2-((methylsulfonyl)oxy)ethyl)benzo[d]thiazol-3-ium (“dimesylate compound”)

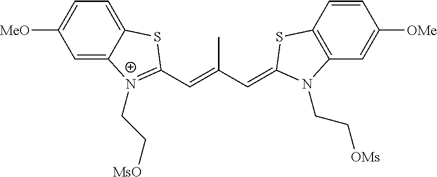 [SMILES: COc1ccc2c(c1)N(CCOS(C)(=O)=O)/C(=C/C(C)=C/c1sc3ccc(OC)cc3[n+]1CCOS(C)(=O)=O)S2]

### and 2-((1E,3Z)-3-(3-(2-Hydroxyethyl)-5-methoxybenzo[d]thiazol-2(3H)-ylidene)-2-methylprop-1-en-1-yl)-5-methoxy-3-(2-((methylsulfonyl)oxy)ethyl)benzo[d]thiazol-3-ium (“monomesylate compound”)

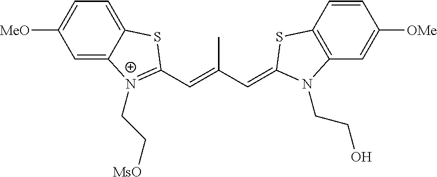 [SMILES: COc1ccc2c(c1)N(CCO)/C(=C/C(C)=C/c1sc3ccc(OC)cc3[n+]1CCOS(C)(=O)=O)S2]

The starting material 3-(2-Hydroxyethyl)-2-((1E,3Z)-3-(3-(2-hydroxyethyl)-5-methoxybenzo[d]thiazol-2(3H)-ylidene)-2-methylprop-1-en-1-yl)-5-methoxybenzo[d]thiazol-3-ium bromide (see Example 2; 0.2 g, 0.36 mmol) was dissolved in 20 mL of pyridine. Methanesulfonyl chloride(0.21 g, 1.8 mmol, 0.14 mL) was added at 0 C . The reaction mass was then stirred at room temperature for another 1.5 h. The completion of the reaction was identified by LCMS. Formation of the mono mesylated and the dimesylated compound was identified. The reaction mass was then quenched with diethyl ether(15 mL).The supernatant solution was removed and the solid were again dissolved in methanol and recrystallized with ether. The resulting material was then purified by semi prep HPLC system to get 50 mg of the dimesylated compound and 60 mg of the monomesylated compound.

Dimesylated Compound: LC-MS: m/z calcd for C₂₆H₃₁Br⁻N⁺2O₈S₄ 706.01, found 627.

¹H NMR (500 MHz, DMSO-d₆): 2.60 (3H, s,CHC(CH₃)CH), 3.16 (6H, s, 2-CH₃SO₂OCH₂), 3.91 (6H, s, 2-OCH), 4.69 (4H, s, 2-NCH₂CH₂OH), 4.90 (4H, s, 2-NCH₂CH₂OH), 6.58 (2H, s, CHC(CH₃)CH), 7.10 (2H, d, 2-Ar-4-CH, J=10 Hz), 7.42 (2H s, 2-Ar-6-CH), 7.92 (2H, d, 2-Ar-7-CH, J=10 Hz)

Monomesylated Compound: LC-MS: m/z calcd for C₂₅H₂₉BrN₂O₆S₃ 628.04, found 549.12. ¹H NMR (500 MHz, DMSO-d₆):2.59 (3H, s,CHC(CH₃)CH), 3.16 (6H, s, 2-CH₃SO₂OCH₂), 3.90 (6H, s, 2-OCH₃), 4.60 (2H, s, NCH₂CH₂OH), 4.67 (2H, s, NCH₂CH₂OH), 4.85(2H, s, NCH₂CH₂OH), 5.16 (2H, s,NCH₂CH₂OH),6.53 (1H, s, CHC(CH₃)CH), 6.65(1H, s, CHC(CH₃)CH), 7.02-7.14 (2H, m, 2-Ar-4-CH), 7.40 (2H, m, 2-Ar-6-CH), 7.93 (2H, m, 2-Ar-7-CH)

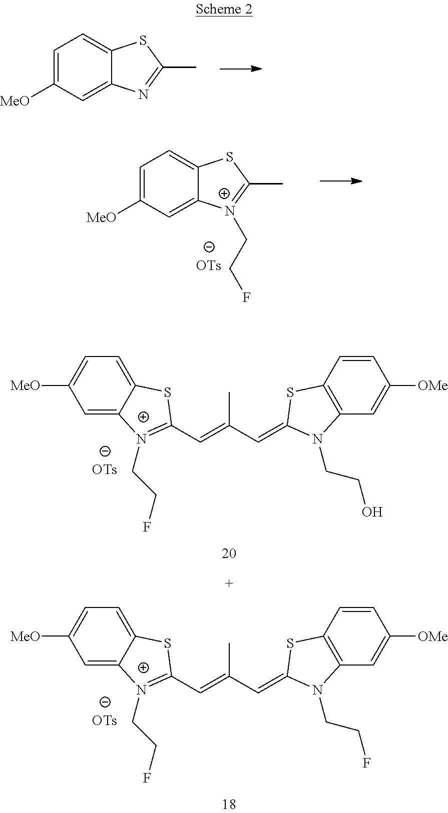 [SMILES: COc1ccc2sc(C)nc2c1 ; COc1ccc2sc(C)[n+](CCF)c2c1 ; COc1ccc2c(c1)N(CCO)/C(=C/C(C)=C/c1sc3ccc(OC)cc3[n+]1CCF)S2 ; COc1ccc2c(c1)N(CCF)/C(=C/C(C)=C/c1sc3ccc(OC)cc3[n+]1CCF)S2]

### Example 4

### Preparation of N-(2-Fluoroethyl)-5-methoxy-2-methylbenzothiazolium tosylate

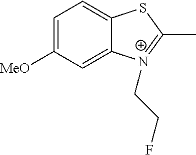

A mixture of 5-methoxy-2-methylbenzothiazole (0.85 g 4.74 mmol) and 2-fluoroethyl 4-methylbenzenesulfonate (3.24 g, 14.84 mmol) was irradiated in microwave at 140 C for 4 hrs. The resulting product was crystallized from ethanol (10 mL) and diethyl ether (100 mL) to yield 1.07 g (95%) as brown solid.

LC-MS: m/z calcd for C₁₁H₁₃FNOS⁺ 226.29, found 226.4

### Example 5

### Preparation of 3-(2-Fluoroethyl)-2-((1E,3Z)-3-(3-(2-hydroxyethyl)-5-methoxybenzo[d]thiazol-2(3H)-ylidene)-2-methylprop-1-en-1-yl)-5-methoxybenzo[d]thiazol-3-ium (20)

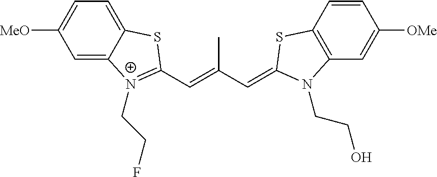 [SMILES: COc1ccc2c(c1)N(CCO)/C(=C/C(C)=C/c1sc3ccc(OC)cc3[n+]1CCF)S2]

A mixture of N-Hydroxyethyl-5-methoxy-2-methylbenzothiazolium bromide (see Example 1; 0.4 g, 1.78 mmol), N-(2-Fluoroethyl)-5-methoxy-2-methylbenzothiazolium tosylate (see Example 4; 0.4 g, 1.76 mmol), triethyl orthoacetate (3.0 mL) in pyridine (35 mL) are heated to 100 C for 16 hrs. The resulting mixture was concentrated under vacuum to dryness. The product is purified by semi prep HPLC and the pure fractions are collected and freeze dried to get 0.072 g of the desired compound as a purple coloured solid.

LC-MS: m/z calcd for C₂₄H₂₆FN₂O₃S₂⁺ 473.60 and found 473.70

¹H NMR (500 MHz, CD₃CN): 2.58 (3H, s, CHC(CH₃)CH), 3.91(3H, s, OCH₃), 3.92 (3H, s, OCH₃), 4.05 (2H, t, NCH₂CH₂OH, J=5 Hz), 4.53 (2H, t, NCH₂CH₂OH, J=5 Hz), 4.65(1H, t, NCH₂CH₂F, J=5 Hz), 4.70(1H, t, NCH₂CH₂F, J=5 Hz) 4.90(1H, t, NCH₂CH₂F, J=5 Hz) 4.99(1H, t, NCH₂CH₂F, J=5 Hz),6.41 (1H, s, CHC(CH₃)CH), 6.61(1H, s, CHC(CH₃)CH), 6.99-7.05 (2H, m, 2-Ar-4-CH), 7.08 (1H, m, 2-Ar-6-CH,J=5 Hz), 7.23 (1H, m, 2-Ar-6-CH, J=5 Hz),7.74 (2H, m, 2-Ar-7-CH,J=5 Hz)

### Example 6

### Preparation of 3-(2-Fluoroethyl)-2-((1E,3Z)-3-(3-(2-fluoroethyl)-5-methoxybenzo[d]thiazol-2(3H)-ylidene)-2-methylprop-1-en-1-yl)-5-methoxybenzo[d]thiazol-3-ium (18)

(35 mg) was isolated from the same reaction set forth in Example 5.

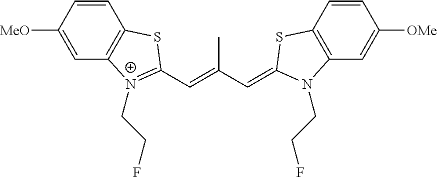 [SMILES: COc1ccc2c(c1)N(CCF)/C(=C/C(C)=C/c1sc3ccc(OC)cc3[n+]1CCF)S2]

LC-MS: m/z calcd for C₂₄H₂₆FN₂O₃S₂⁺ 475.59 and found 475.6 (M+)

¹H NMR (300 MHz, DMSO d6): 2.58 (3H, s, CHC(CH₃)CH), 3.91(6H, s, OCH₃), 4.85(4H, Bs, NCH₂CH₂F) 4.93(2H, Bs, NCH₂CH₂F) 5.00(2H, Bs, NCH₂CH₂F), 6.58(2H, s, CHC(CH₃)CH), 7.09 (2H, dd, 2-Ar-6-CH,J=3.9 Hz), 7.4 (2H, Bs, 2-Ar-6-CH,),7.95 (2H, d, 2-Ar-7-CH,J=9 Hz)

### Example 7.

### Aggregation of tau and Aβ

Tau protein (Tau-412 with 2 nucleotide-binding motifs and 4 repeat binding regions, 42.9 kDa, 1 mg/ml, Stratech) was incubated in the presence of heparin (0.1 mg/ml) for 72 h at 37° C. with continuous shaking to generate tau aggregates.

Aβ₁₋₄₀ protein (4.3 kDa, 1 mg/ml, Invitrogen) was incubated at room temperature (RT) for 72 h with continuous shaking to generate Aβ₁₋₄₀ aggregates.

In vitro Binding Assay

The two test compounds 18 (see Example 6) and 20 (see Example 5), were dissolved and serially diluted in DMSO. Samples with aggregated tau and Aβ₁₋₄₀ (10 μg/sample) were incubated with a serial dilution of each test compound (concentration range 1 nM-100 μM) in 96-well plates for 1 h at RT. Following incubation, samples were transferred to spin column plates (Thermo Fisher) and then centrifugated for 2 min at 1,500×g to remove free compound from the samples. Finally, the samples were transferred to black 96-well plates, and the fluorescence intensity measured at 485-612 nM using a Tecan plate reader. All experiments were performed in triplicate. Kd values for each compound was calculated by fitting the data to a non-linear binding curve using Prism (GraphPad).

### Tissue Binding Assay

Frozen tissue chucks from entorhinal cortex from patients with Alzheimer's disease and healthy subjects were obtained from Tissue Solution (Clydebank, United Kingdom) The tissue was cryosectioned, post-fixed in neutral buffered formalin, and the tissue autofluorescence was quenched by incubation of tissue in 0.25% KMnO4 in phosphate buffered saline. Adjacent tissue sections were labelled with each compound (100 μM, 30 min incubation at RT), followed by overnight incubation with primary antibodies directed against either neurofibrillary tangles (mouse anti-tau, clone ATB, 1:20 dilution, Innogenetics) or Aβ plaques (mouse anti-Aβ, clone 4G8, 1:50 dilution, Cambridge Bioscience) at 4° C. Finally the tissue sections were incubated with fluorescence secondary antibodies (ALEXA488 or ALEXA564 goat anti-mouse, dilution 1:1000, Invitrogen) for 1 h at RT and then mounted in Vectashield mounting media with Dapi to counterstain cell nuclei. Pictures were captured using a Nikon DiRS camera connected to a Leica microscope.

### Results

The results demonstrate that the tested compounds bind to tau aggregates with Kds in the low nanomolar range, whereas Kds to Aβ₁₋₄₀ aggregates were significantly higher. Thus, both 18 and 20 are highly selective for aggregated tau compared to Aβ₁₋₄₀ aggregates.

| | Kd | | Selectivity |
|---|---|---|---|
| Compounds | Tau | Aβ₁₋₄₀ | Tau: Aβ₁₋₄₀ |
| 18 | 0.88 ± 0.90 nM | 29.8 ± 21.3 μM | 33,863 |
| 20 | 0.75 ± 0.59 nM | 15.6 ± 8.1 μM | 20,800 |

### Example 8

### Preparation of [18F]3-(2-Fluoroethyl)-2-((1E,3Z)-3-(3-(2-hydroxyethyl)-5-methoxybenzo[d]thiazol-2(3H)-ylidene)-2-methylprop-1-en-1-yl)-5-methoxybenzo[d]thiazol-3-ium ([¹⁸F]9)

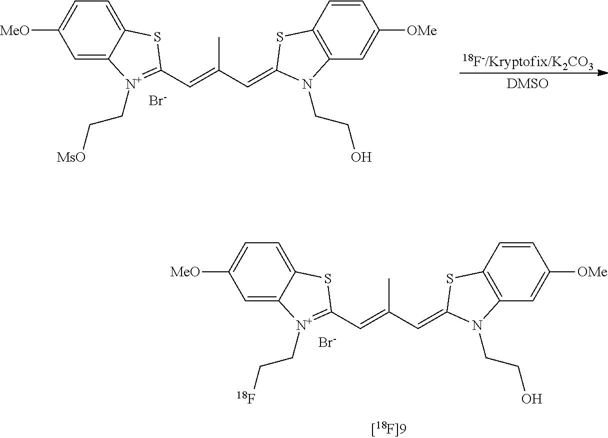 [SMILES: COc1ccc2c(c1)N(CCO)/C(=C/C(C)=C/c1sc3ccc(OC)cc3[n+]1CCOS(C)(=O)=O)S2 ; [Br-] ; COc1ccc2c(c1)N(CCO)/C(=C/C(C)=C/c1sc3ccc(OC)cc3[n+]1CC[18F])S2 ; [Br-]]

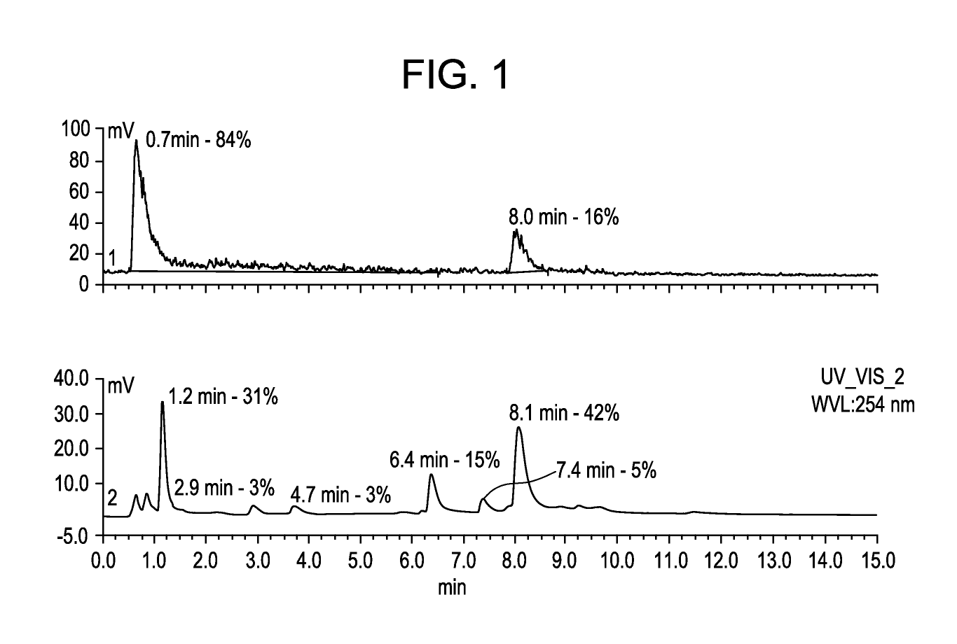

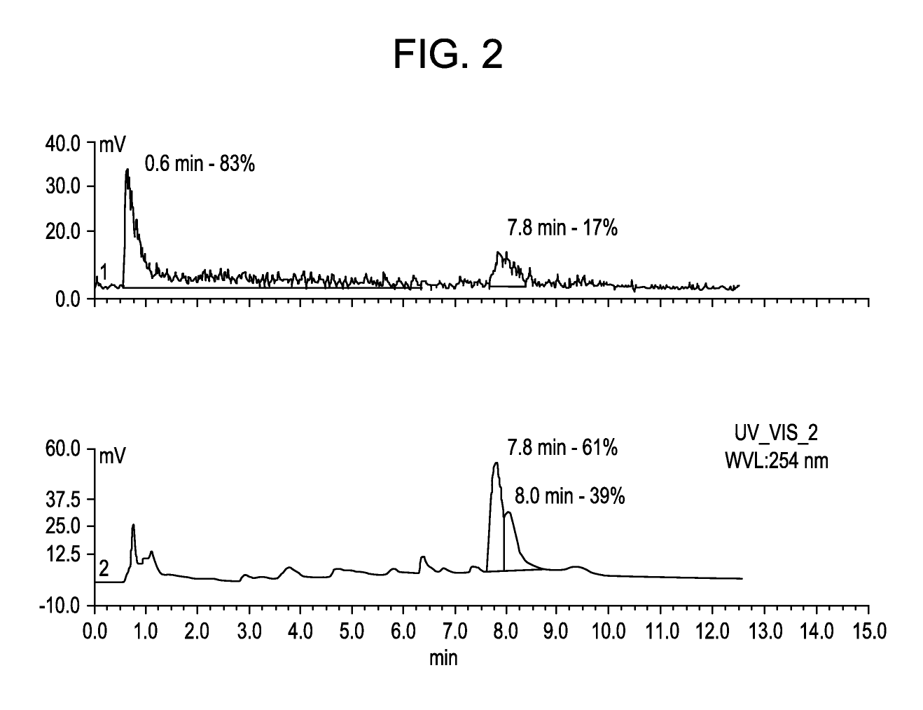

Fluorine-18 was obtained from a cyclotron by irradiating enriched [¹⁸O]H₂O using the ¹⁸O(p,n)¹⁸F reaction. An aqueous solution of [¹⁸]fluoride (0.2 mL, 108 MBq) was added to a Wheaton vial (3 mL) containing potassium carbonate (1 mg, 7.2 μmol), Kryptofix™ (5 mg, 13.2 μmol), and acetonitrile (1 mL). The vial was heated to 100° C. and the solvent removed using a stream of nitrogen. Anhydrous acetonitrile (0.5 mL) was added and the evaporation repeated. This azeotropic drying step was repeated two times. After cooling to room temperature, a solution 2-((1E,3Z)-3-(3-(2-Hydroxyethyl)-5-methoxybenzo[d]thiazol-2(3H)-ylidene)-2-methylprop-1-en-1-yl)-5-methoxy-3-(2-((methylsulfonyl)oxy)ethyl)benzo[d]thiazol-3-ium (see Example 3; 2 mg, 3.2 μmol) in anhydrous dimethylsulfoxide (0.2 mL) was added. The reaction mixture was heated for 15 minutes at 80 ° C. and analysed by analytical HPLC (column: Luna, Phenomenex, C18(2), 50×4.6 mm, 3 μm; solvent A: ammonium acetate buffer, 20 mM, pH 4.3, solvent B: acetonitrile; gradient: 20 to 80% B in 15 min, flow rate: 1 mL/min, UV wave-length: 254 nm). The HPLC analysis showed a 16% labelling efficiency to form [¹⁸F]9 (FIG. 1) and co-elution with stable ¹⁹F-9 reference material (FIG. 2).

### Example 9

### Preparation of [18F]3-(2-Fluoroethyl)-2-((1E,3Z)-3-(3-(2-hydroxyethyl)-5-methoxybenzo[d]thiazol-2(3H)-ylidene)-2-methylprop-1-en-1-yl)-5-methoxybenzo[d]thiazol-3-ium ([¹⁸F]9)

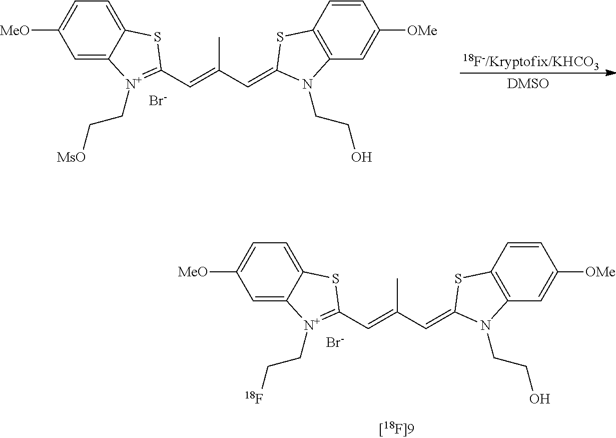 [SMILES: COc1ccc2c(c1)N(CCO)/C(=C/C(C)=C/c1sc3ccc(OC)cc3[n+]1CCOS(C)(=O)=O)S2 ; [Br-] ; COc1ccc2c(c1)N(CCO)/C(=C/C(C)=C/c1sc3ccc(OC)cc3[n+]1CC[18F])S2 ; [Br-]]

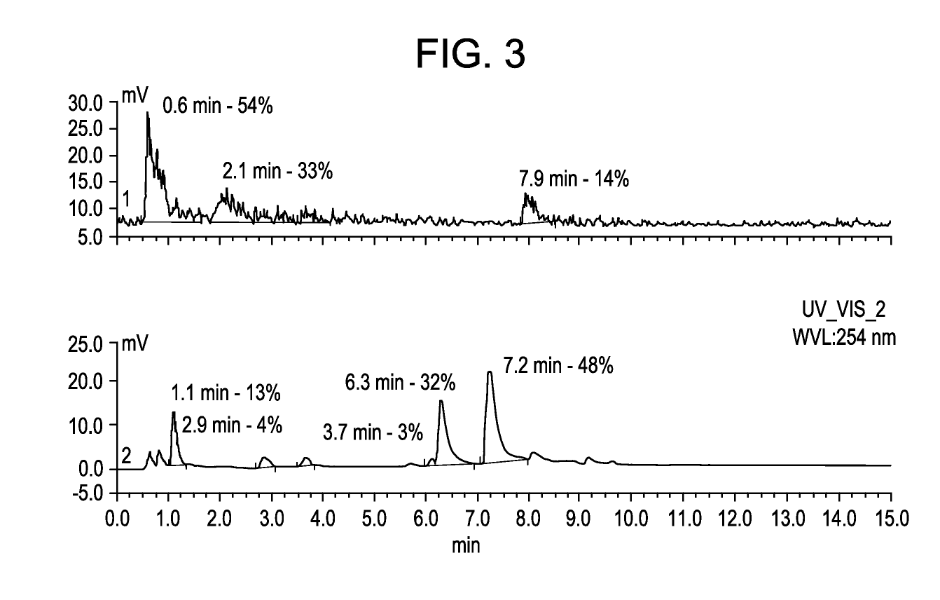

Compound [¹⁸F]9 was prepared following the procedure set forth in Example 8 except using potassium bicarbonate (1 mg, 10 μmol) instead of potassium carbonate. Analysis of the reaction mixture by radio-HPLC showed a labelling efficiency of 14% (FIG. 3).

All patents, journal articles, publications and other documents discussed and/or cited above are hereby incorporated by reference.

What is claimed is:

1. A radiolabeled compound of Formula (I): 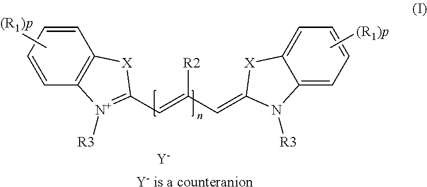
wherein:
R₁ are each independently H, alkyl, alkoxy, hydroxyalkyloxy, or hydroxy poly(oxoethylene)oxy;
p are each independently an integer from 1-4;
X are each independently N, O or S;
R2 is H or alkyl;
R3 are each independently H, alkyl, hydroxyalkyl or hydroxy poly(oxoethylene);
n is an integer from 1-5; and
Y is a counteranion;
and wherein at least one of R1, R2, and R3 contains a radionuclide.

2. A radiolabeled compound of Formula (Ia): 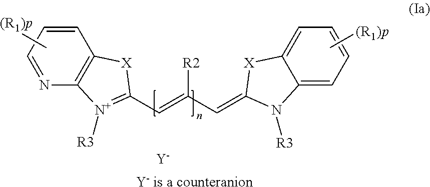
wherein:
R₁ are each independently H, alkyl, alkoxy, hydroxyalkyloxy, or hydroxy poly(oxoethylene)oxy;
p are each independently an integer from 1-4;
X are each independently N, O or S;
R2 is H or alkyl;
R3 are each independently H, alkyl, hydroxyalkyl or hydroxy poly(oxoethylene);
n is an integer from 1-5; and
Y is a counteranion;
and wherein at least one of R1, R2, and R3 contains a radionuclide.

3. A radiolabeled compound of Formula (II): 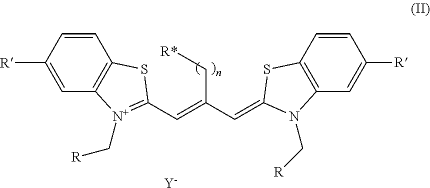
wherein:
R are each independently H, alkyl, hydroxyalkyl or hydroxy poly(oxoethylene);
R′ are each independently H, alkyl, alkoxy, hydroxyalkyloxy, or hydroxy poly(oxoethylene)oxy;
n is an integer from 1-6;
R* is a radionuclide; and
Y⁻ is a counteranion.

4. A radiolabeled compound of Formula (IIa): 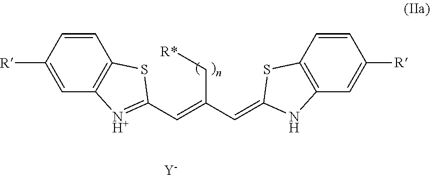
wherein R′, n, R*, and Y⁻ are each as defined for a compound of Formula (II) of claim 3.

5. A radiolabeled compound of Formula (III): 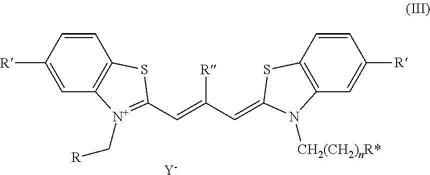
wherein:
R is H, alkyl, hydroxyalkyl or hydroxy poly(oxoethylene);
R′ are each independently H, alkyl, alkoxy, hydroxyalkyloxy, or hydroxy poly(oxoethylene)oxy;
R″ is H or alkyl;
R* is a radionuclide;
n is an integer from 1-10; and
Y⁻ is a counteranion.

6. A radiolabeled compound of Formula (IV): 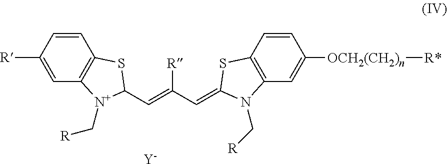
wherein:
R are each independently H, alkyl, hydroxyalkyl or hydroxy poly(oxoethylene);
R′ is H, alkyl, alkoxy, hydroxyalkyloxy, or hydroxy poly(oxoethylene)oxy;
R″ is H or alkyl;
R* is a radionuclide;
n is an integer from 1-10; and
Y⁻ is a counteranion.

7. A radiolabeled compound of Formula (IVa): 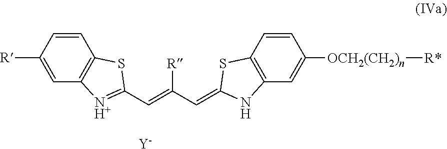
wherein R′, R″, R*, n and Y⁻ are each as defined for a compound of Formula (IV) of claim 6.

8. A pharmaceutical composition comprising a compound according to any one of claims 1-7 and a pharmaceutically acceptable carrier or excipient.

9. A method of making a compound of according to any one of claims 1-7.

10. A method of imaging using a compound according to any one of claims 1-7 or a pharmaceutical composition thereof.

11. A method of detecting tau aggregates in vitro and/or in vivo using a compound according to any one of claims 1-7 or a pharmaceutical composition thereof.

12. A compound of the following formula:

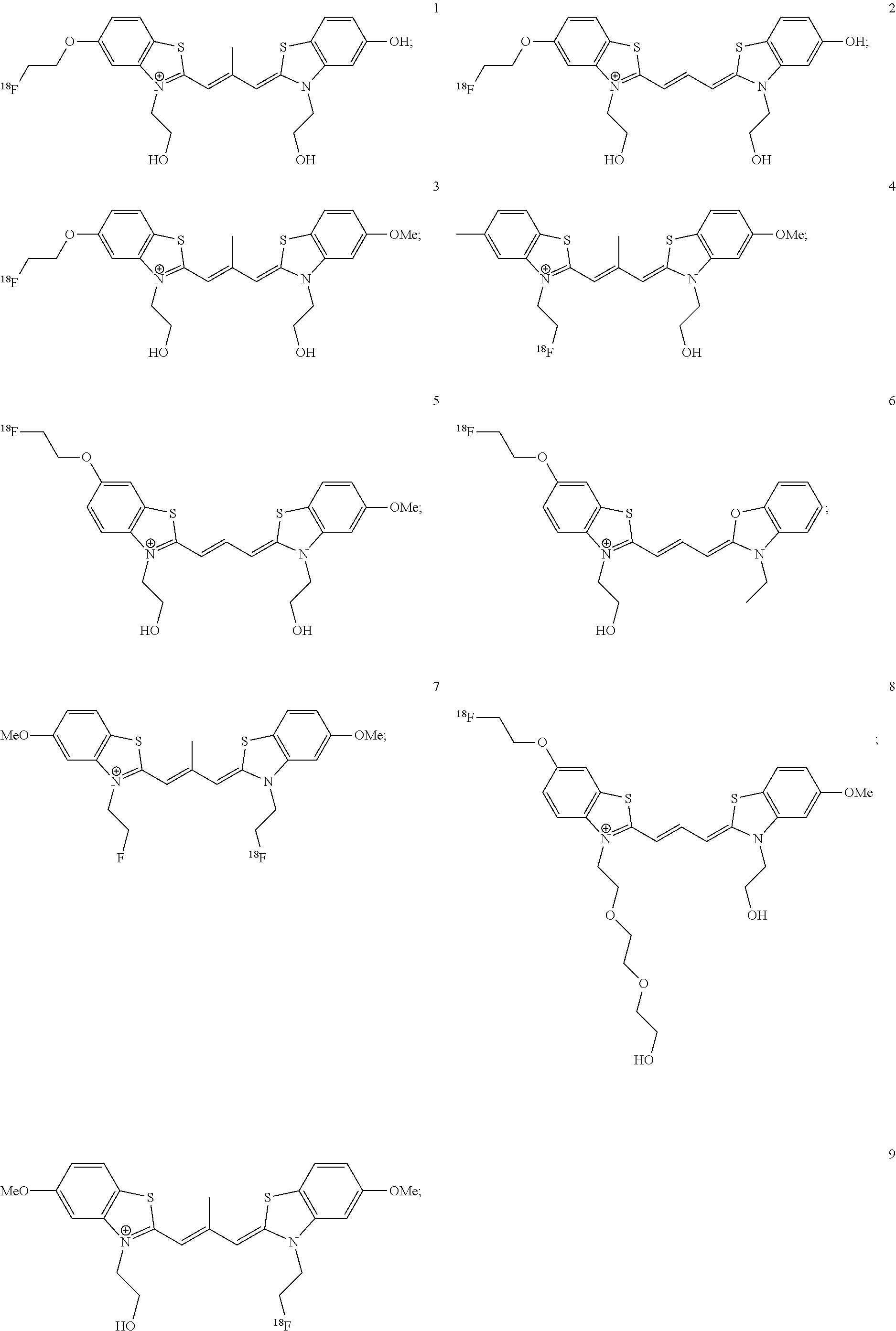

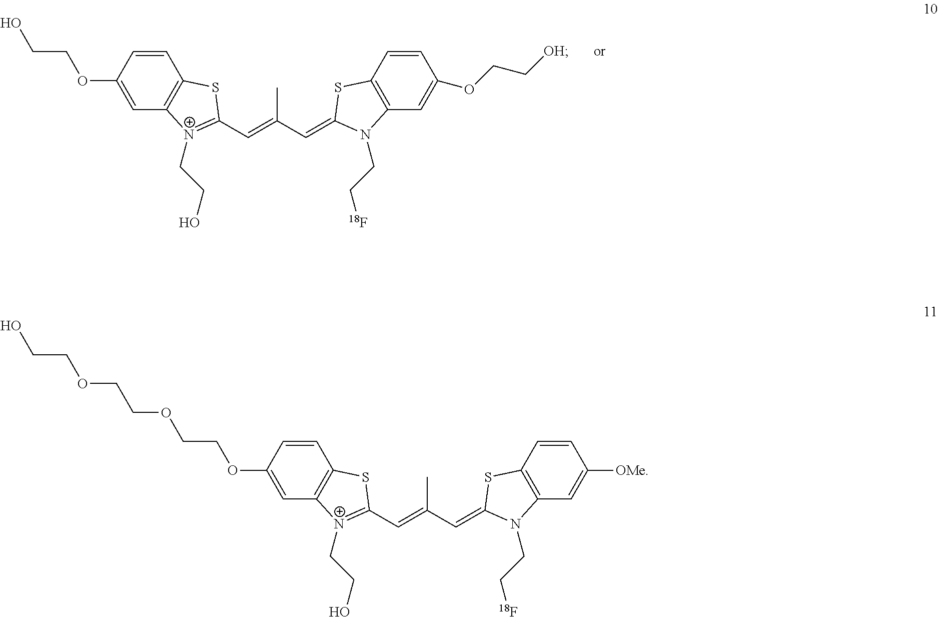 [SMILES: CC(/C=C1\Sc2ccc(OCCO)cc2N1CC[18F])=C\c1sc2ccc(OCCO)cc2[n+]1CCO ; COc1ccc2c(c1)N(CC[18F])/C(=C/C(C)=C/c1sc3ccc(OCCOCCOCCO)cc3[n+]1CCO)S2]

13. A pharmaceutical composition comprising a compound of claim 12 and a pharmaceutically acceptable carrier or excipient.

14. A method of imaging using a compound of claim 12 or a pharmaceutical composition thereof.

15. A method of detecting tau aggregates in vitro and/or in vivo using a compound according to claim 12 or a pharmaceutical composition thereof.

16. A compound of the following formula:

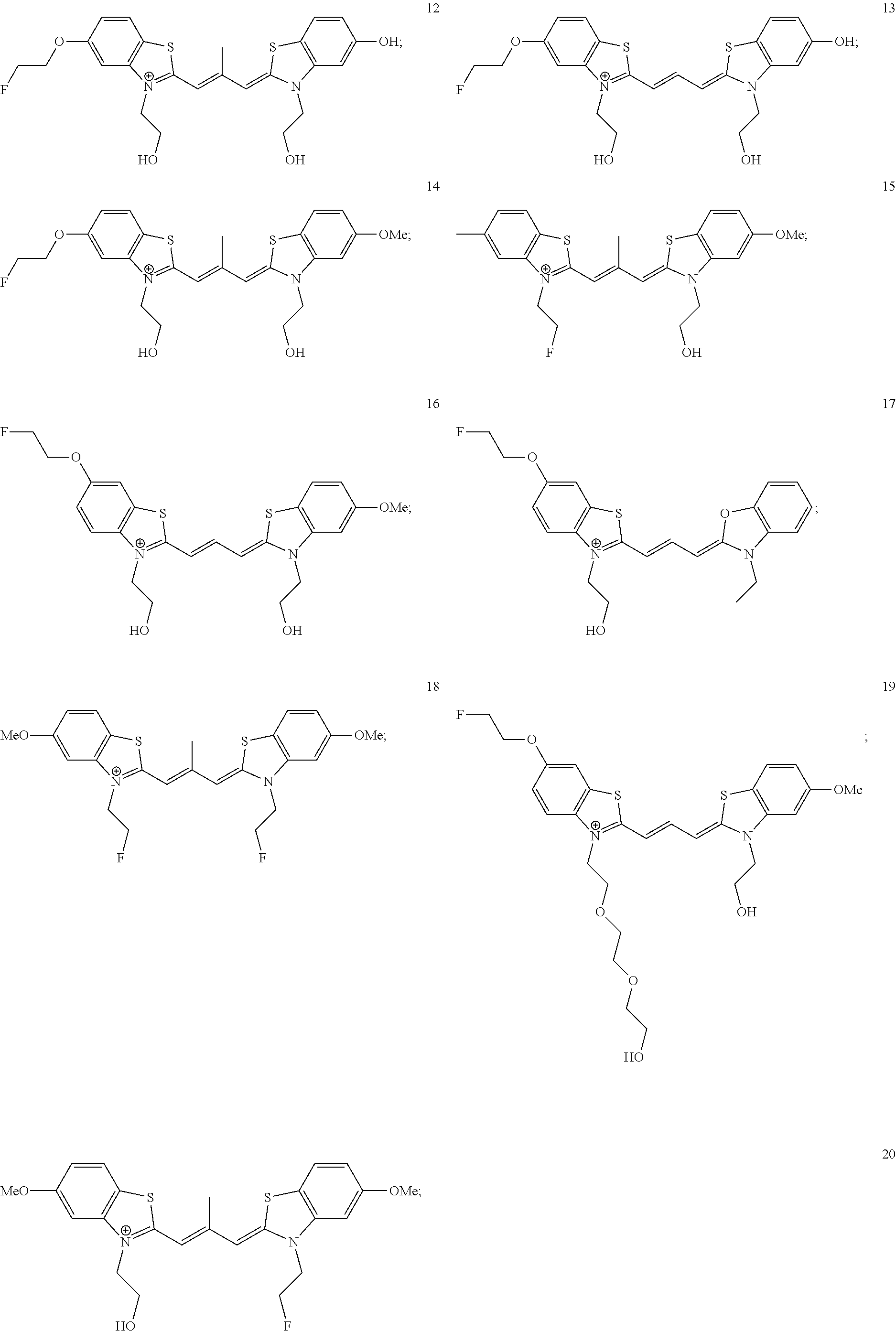

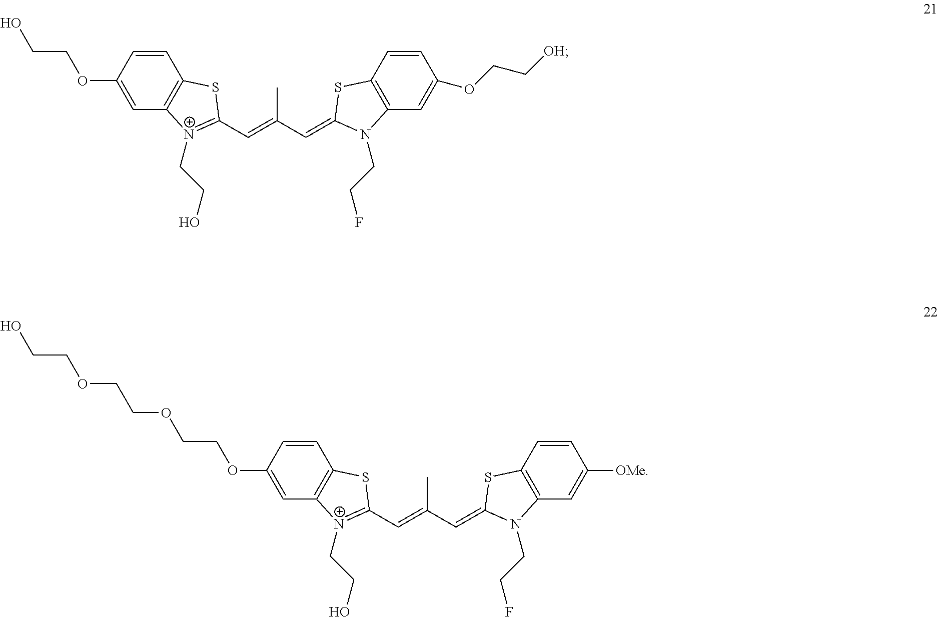 [SMILES: CC(/C=C1\Sc2ccc(OCCO)cc2N1CCF)=C\c1sc2ccc(OCCO)cc2[n+]1CCO ; COc1ccc2c(c1)N(CCF)/C(=C/C(C)=C/c1sc3ccc(OCCOCCOCCO)cc3[n+]1CCO)S2]

17. A pharmaceutical composition comprising a compound of claim 16 and a pharmaceutically acceptable carrier or excipient.
